(19) United States
(12) Patent Application Publication
(10) Pub. No.: US 2021/0004961 A1
(43) Pub. Date: Jan. 7, 2021

(54) IMAGE PROCESSING APPARATUS, CAPSULE ENDOSCOPE SYSTEM, METHOD OF OPERATING IMAGE PROCESSING APPARATUS, AND COMPUTER-READABLE STORAGE MEDIUM

(71) Applicant: OLYMPUS CORPORATION, Tokyo (JP)

(72) Inventors: Masaki TAKAHASHI, Saitama-shi (JP); Hironao KAWANO, Tokyo (JP); Takeshi NISHIYAMA, Tokyo (JP)

(73) Assignee: OLYMPUS CORPORATION, Tokyo (JP)

(21) Appl. No.: 17/025,225

(22) Filed: Sep. 18, 2020

Related U.S. Application Data
(63) Continuation of application No. PCT/JP2018/032918, filed on Sep. 5, 2018.

(30) Foreign Application Priority Data
Mar. 27, 2018 (JP) 2018-060859

(51) Int. Cl. G06T 7/00; G06K 9/78; G06T 7/70; A61B 1/04; A61B 1/00

(52) U.S. Cl. CPC G06T 7/0014; G06K 9/78; G06T 7/70; A61B 1/041; G06T 2207/30092; G06K 2209/05; G06T 2207/10068; G06T 2207/30096; G06T 2207/30028; A61B 1/00045

(57) ABSTRACT

An image processing apparatus includes: an identification circuit configured to calculate a characteristic of each of a plurality of image groups captured when a capsule endoscope is introduced into a subject multiple times, each image group being a group of images that are captured each time the capsule endoscope is introduced into the subject, and identify, based on the calculated characteristic, a first region or a second region in each image group, the first region being a region that does not include an image of the subject captured by the capsule endoscope, the second region being a region that is regarded as not including the captured image of the subject; and a first specifying circuit configured to specify at least one section of the subject in the image groups, the at least one section including the first region or the second region.

### CROSS-REFERENCE TO RELATED APPLICATIONS

This application is a continuation of PCT international application Ser. No. PCT/JP2018/032918, filed on Sep. 5, 2018 which designates the United States, incorporated herein by reference, and which claims the benefit of priority from Japanese Patent Applications No. 2018-060859, filed on Mar. 27, 2018, incorporated herein by reference.

### BACKGROUND

### 1. Technical Field

The present disclosure relates to an image processing apparatus, a capsule endoscope system, a method of operating an image processing apparatus, and a computer-readable storage medium.

### 2. Related Art

In the field of endoscopes, a capsule endoscope that is introduced into a subject to capture an image has been developed. The capsule endoscope has an imaging function and a wireless communication function inside a capsule-shaped casing formed to have a size that enables introduction into the gastrointestinal tract of a subject. The capsule endoscope is swallowed by the subject and thereafter captures an image while moving inside the gastrointestinal tract by a peristaltic motion or the like, and sequentially generates and wirelessly transmits an image (hereinafter, also referred to as in-vivo image) of an internal portion of an organ of the subject (see, for example, JP 2012-228346 A). The wirelessly transmitted image is received by a receiving device provided outside the subject. Further, the received image is fetched to an image processing apparatus such as a workstation and subjected to predetermined image processing. As a result, the in-vivo image of the subject can be displayed as a still image or a moving image on a display device connected to the image processing apparatus.

When finding a lesion such as a bleeding source using the capsule endoscope, in a case where the lesion cannot be found in a single examination, the capsule endoscope may be introduced into the same subject multiple times for examination.

### SUMMARY

In some embodiments, provided is an image processing apparatus that performs image processing on an image captured by a capsule endoscope introduced into a subject. The image processing apparatus includes: an identification circuit configured to calculate a characteristic of each of a plurality of image groups captured when the capsule endoscope is introduced into the subject multiple times, each image group being a group of images that are captured each time the capsule endoscope is introduced into the subject, and identify, based on the calculated characteristic, a first region or a second region in each image group, the first region being a region that does not include an image of the subject captured by the capsule endoscope, the second region being a region that is regarded as not including the captured image of the subject; and a first specifying circuit configured to specify at least one section of the subject in the plurality of image groups, the at least one section including the first region or the second region.

In some embodiments, a capsule endoscope system includes: the image processing apparatus; and the capsule endoscope.

In some embodiments, provided is a method of operating an image processing apparatus that performs image processing on an image captured by a capsule endoscope introduced into a subject. The method includes: calculating, by an identification circuit, a characteristic of each of a plurality of image groups captured when the capsule endoscope is introduced into the subject multiple times, each image group being a group of images that are captured each time the capsule endoscope is introduced into the subject; identifying, based on the calculated characteristic, a first region or a second region in each of the plurality of image groups, the first region being a region that does not include an image of the subject captured by the capsule endoscope, the second region being a region that is regarded as not including the captured image of the subject; and specifying, by a first specifying circuit, at least one section of the subject in each of the plurality of image groups, the at least one section including the first region or the second region.

In some embodiments, provided is a non-transitory computer-readable recording medium on which an executable program is recorded. The program instructs an image processing apparatus that performs image processing on an image captured by a capsule endoscope introduced into a subject to execute: calculating, by an identification circuit, a characteristic of each of a plurality of image groups captured when the capsule endoscope is introduced into the subject multiple times, each image group being a group of images that are captured each time the capsule endoscope is introduced into the subject, identifying, based on the calculated characteristic, a first region or a second region in each of the plurality of image groups, the first region being a region that does not include an image of the subject captured by the capsule endoscope, the second region being a region that is regarded as not including the captured image of the subject; and specifying, by a first specifying circuit, at least one section of the subject in each of the plurality of image groups, the at least one section including the first region or the second region.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating a schematic configuration of a capsule endoscope system including an image processing apparatus according to a first embodiment;

FIG. 2 is a block diagram illustrating the image processing apparatus illustrated in FIG. 1;

FIG. 3 is a flowchart illustrating an operation of the image processing apparatus illustrated in FIG. 2;

FIG. 4 is a flowchart illustrating identification processing illustrated in FIG. 3;

FIG. 5 is a diagram illustrating an example of an image displayed on a display device;

FIG. 6 is a block diagram illustrating an image processing apparatus according to Modified Example 1-1;

FIG. 7 is a flowchart illustrating identification processing of the image processing apparatus illustrated in FIG. 6;

FIG. 8 is a block diagram illustrating an image processing apparatus according to Modified Example 1-2;

FIG. 9 is a flowchart illustrating an operation of the image processing apparatus illustrated in FIG. 8;

FIG. 10 is a diagram illustrating a reciprocating image group;

FIG. 11 is a diagram illustrating a state in which an overlapping section is specified from the reciprocating image group;

FIG. 12 is a diagram illustrating a state in which an image processing apparatus according to a second embodiment specifies overlapping sections;

FIG. 13 is a diagram illustrating a state in which an image processing apparatus according to Modified Example 2-1 specifies overlapping sections;

FIG. 14 is a diagram illustrating a state in which an image processing apparatus according to a third embodiment specifies an overlapping section;

FIG. 15 is a diagram illustrating a state in which an image processing apparatus according to Modified Example 3-1 specifies an overlapping section;

FIG. 16 is a diagram illustrating a state in which an image processing apparatus according to Modified Example 3-2 specifies an overlapping section;

FIG. 17 is a block diagram illustrating an image processing apparatus according to a fourth embodiment;

FIG. 18 is a block diagram illustrating an image processing apparatus according to Modified Example 4-1;

FIG. 19 is a diagram illustrating an example of an image displayed on the display device;

FIG. 20 is a diagram illustrating a state in which reference positions match each other;

FIG. 21 is a diagram illustrating a state in which a non-captured proportion is displayed;

FIG. 22 is a diagram illustrating a state in which a captured proportion is displayed;

FIG. 23 is a diagram illustrating a state in which distance bars are displayed side by side; and

FIG. 24 is a diagram illustrating a state in which a distance bar is hidden.

### DETAILED DESCRIPTION

Hereinafter, embodiments will be described with reference to the accompanying drawings.

### First Embodiment

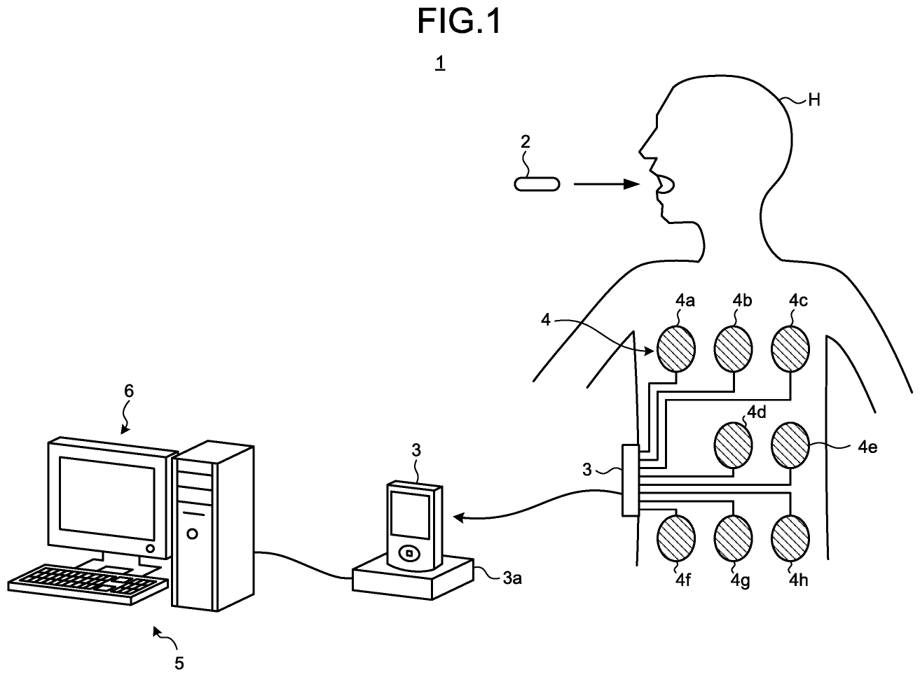

FIG. 1 is a schematic diagram illustrating a schematic configuration of a capsule endoscope system including an image processing apparatus according to a first embodiment. A capsule endoscope system 1 illustrated in FIG. 1 includes

a capsule endoscope 2 that is introduced into a subject H such as a patient, generates an image obtained by capturing the inside of the subject H, and wirelessly transmits the generated image, a receiving device 3 that receives the image wirelessly transmitted from the capsule endoscope 2 via a receiving antenna unit 4 attached to the subject H, an image processing apparatus 5 that acquires the image from the receiving device 3, performs predetermined image processing on the acquired image, and displays the processed image, and a display device 6 that displays the image of the inside of the subject H, or the like in response to an input from the image processing apparatus 5.

The capsule endoscope 2 is constituted by an image sensor such as a charge coupled device (CCD) or a complementary metal oxide semiconductor (CMOS). The capsule endoscope 2 is a capsule type endoscope device formed to have a size that enables introduction into an organ of the subject H. The capsule endoscope 2 is introduced into the organ of the subject H by oral insertion or the like, and sequentially captures in-vivo images while moving inside the organ by a peristaltic motion or the like, and maintaining a predetermined frame rate. Then, the images generated by the capturing are sequentially transmitted by an embedded antenna or the like.

The receiving antenna unit 4 includes a plurality of (eight in FIG. 1) receiving antennas 4a to 4h. Each of the receiving antennas 4a to 4h is implemented by a loop antenna, for example, and disposed at a predetermined position on an external surface of the subject H (for example, a position corresponding to each organ inside the subject H, the organ being a region through which the capsule endoscope 2 passes).

The receiving device 3 receives the image wirelessly transmitted from the capsule endoscope 2 via these receiving antennas 4a to 4h, performs predetermined processing on the received image, and stores the image and information regarding the image in an embedded memory. The receiving device 3 may include a display unit that displays a state of reception of the image wirelessly transmitted from the capsule endoscope 2, and an input unit such as an operation button to operate the receiving device 3. Further, the receiving device 3 includes a general-purpose processor such as a central processing unit (CPU), or a special-purpose processor such as various arithmetic operation circuits that perform specific functions, such as an application specific integrated circuit (ASIC) and a field programmable gate array (FPGA).

The image processing apparatus 5 performs image processing on each of a plurality of image groups captured by introducing the capsule endoscope 2 into the same subject H multiple times. Each image group is a group of in-vivo images of the subject H that are arranged in time series, the in-vivo images being captured by the capsule endoscope 2 introduced into the subject H until the capsule endoscope 2 is pulled out of the body of the subject H. The image processing apparatus 5 is implemented by a workstation or personal computer including a general- purpose processor such as a CPU, or a special-purpose processor such as various arithmetic operation circuits that execute a certain function, such as an ASIC and an FPGA. The image processing apparatus 5 fetches the image and the information regarding the image, the image and the information being stored in the memory of the receiving device 3, performs predetermined image processing, and displays the image on the screen. Note that FIG. 1 illustrates a configuration in which a cradle 3a is connected to a universal serial bus (USB) port of the image processing apparatus 5, the receiving device 3 is connected to the image processing apparatus 5 by setting the receiving device 3 in the cradle 3a, and an image and information regarding the image are transferred from the receiving device 3 to the image processing apparatus 5. Note that a configuration in which an image and information regarding the image are wirelessly transmitted from the receiving device 3 to the image processing apparatus 5 via an antenna or the like may also be possible.

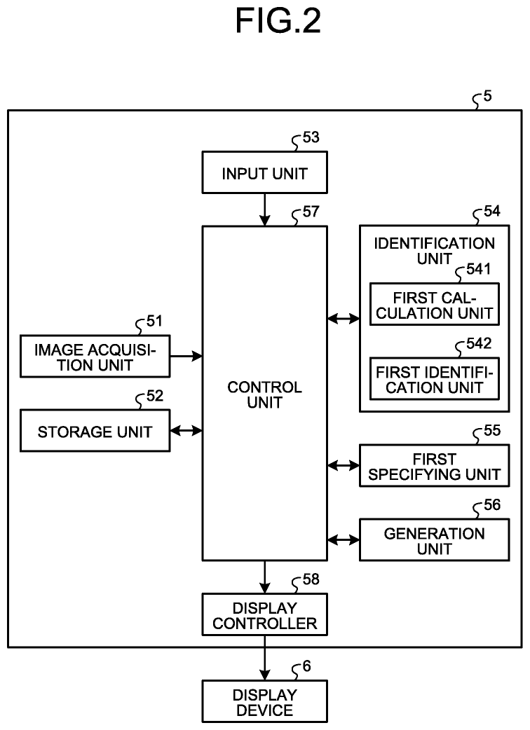

FIG. 2 is a block diagram illustrating the image processing apparatus illustrated in FIG. 1. The image processing apparatus 5 illustrated in FIG. 2 includes an image acquisition unit 51, a storage unit 52, an input unit 53, an identification unit 54, a first specifying unit 55, a generation unit 56, a control unit 57, and a display controller 58.

The image acquisition unit 51 acquires an image to be processed from the outside. Specifically, the image acquisition unit 51 fetches, under the control of the control unit 57, an image (an image group including a plurality of in-vivo images captured (acquired) in time series by the capsule endoscope 2) stored in the receiving device 3 set in the cradle 3a, via the cradle 3a connected to the USB port. Further, the image acquisition unit 51 also causes the storage unit 52 to store the fetched image group via the control unit 57.

The storage unit 52 is implemented by various IC memories such as a flash memory, a read only memory (ROM), and a random access memory (RAM), a hard disk that is built-in or connected by a data communication terminal, or the like. The storage unit 52 stores the image group transferred from the image acquisition unit 51 via the control unit 57. Further, the storage unit 52 stores various programs (including an image processing program) executed by the control unit 57, information required for processing performed by the control unit 57, or the like.

The input unit 53 is implemented with input devices such as a keyboard, a mouse, a touch panel, and various switches, and outputs, to the control unit 57, input signals generated in response to an external operation on these input devices.

The identification unit 54 calculates a characteristic of each of the plurality of image groups to identify a region of the subject H that is not captured by the capsule endoscope 2, in each of the plurality of image groups on the basis of the characteristic. Specifically, the identification unit 54 includes a first calculation unit 541 that calculates, as a characteristic, the amount of a specific region in each image of each of the plurality of image groups, and a first identification unit 542 that identifies the region of the subject H that is not captured by the capsule endoscope 2 on the basis of the amount of the specific region calculated by the first calculation unit 541. The specific region is a region including a captured image of, for example, a bubble or residue in the gastrointestinal tract, or noise caused by a poor state of communication between the capsule endoscope 2 and the receiving device 3. Further, the specific region may include a region including a captured image of bile. Further, the identification unit 54 may identify a blurred image caused by fast movement of the capsule endoscope 2. Alternatively, a configuration in which a user can select a specific target to be included in the specific region by setting. The identification unit 54 includes a general- purpose processor such as a CPU or a special-purpose processor such as various arithmetic operation circuits that perform specific functions, such as an ASIC and an FPGA.

Note that the specific region can be detected by applying a known method. For example, as disclosed in JP 2007-313119 A, it is allowable to detect a bubble region by detecting a match between a bubble model to be set on the basis of a feature of a bubble image, such as an arc-shaped protruding edge due to illumination reflection, existing at a contour portion of a bubble or inside the bubble, and an edge extracted from an intraluminal image. Alternatively, as disclosed in JP 2012-143340 A, it is allowable to detect a residue candidate region, that is assumed to be a non-mucosa region, on the basis of color feature data based on each pixel value, and to discern whether or not the residue candidate region is a mucosa region on the basis of a positional relationship between the residue candidate region and the edge extracted from the intraluminal image.

The first specifying unit 55 specifies a section of the subject H in which the region identified by the identification unit 54 in the image group overlaps each other between the plurality of image groups. However, the first specifying unit 55 may specify at least one section of the subject H in which the region is included in one of the plurality of image groups. Specifically, the first specifying unit 55 may specify a section of the subject H in which the region identified by the identification unit 54 is included in any one of the plurality of image groups. Further, the first specifying unit 55 may specify a section of the subject H in which an overlapping proportion of the region of the image group identified by the identification unit 54 overlapping each other between the plurality of image groups is equal to or more than a predetermined value. The first specifying unit 55 includes a general-purpose processor such as a CPU or a special-purpose processor such as various arithmetic operation circuits that perform specific functions, such as an ASIC and an FPGA.

The generation unit 56 generates information regarding a position of the section specified by the first specifying unit 55. The information generated by the generation unit 56 is, for example, a distance from a reference position of the subject H to the section. However, the generation unit 56 may generate information regarding a position of the section specified by the first specifying unit 55 for at least one section. Further, the information generated by the generation unit 56 may include a distance from the reference position of the subject H to a position where the section ends, a distance from the reference position of the subject H to an intermediate position of the section, the length of the section, and the like. The generation unit 56 includes a general-purpose processor such as a CPU or a special-purpose processor such as various arithmetic operation circuits that perform specific functions, such as an ASIC and an FPGA.

The control unit 57 reads a program (including the image processing program) stored in the storage unit 52 and controls an overall operation of the image processing apparatus 5 according to the program. The control unit 57 includes a general-purpose processor such as a CPU or a special-purpose processor such as various arithmetic operation circuits that perform specific functions, such as an ASIC and an FPGA. Alternatively, the control unit 57 may include the identification unit 54, the first specifying unit 55, the generation unit 56, the display controller 58, and the like, and one CPU and the like.

The display controller 58 controls display performed by the display device 6 under the control of the control unit 57. Specifically, the display controller 58 controls display performed by the display device 6 by generating and outputting a video signal. The display controller 58 causes the display device 6 to display the information generated by the generation unit. The display controller 58 includes a general-purpose processor such as a CPU or a special-purpose processor such as various arithmetic operation circuits that perform specific functions, such as an ASIC and an FPGA.

The display device 6 is implemented by a liquid crystal display, an organic electroluminescence (EL) display, or the like, and displays a display screen such as an in-vivo image under the control of the display controller 58.

Next, an operation of the image processing apparatus 5 will be described. Hereinafter, processing for two image groups including first and second image groups will be described. However, the number of image groups is not particularly limited as long as it is plural.

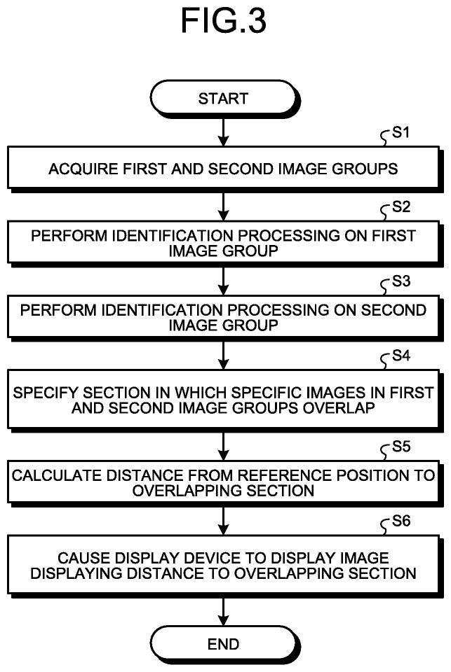

FIG. 3 is a flowchart illustrating an operation of the image processing apparatus illustrated in FIG. 2. As illustrated in FIG. 3, the first and second image groups stored in the storage unit 52 are acquired (Step S1). Here, as the image groups, the first and second image groups that are respectively captured by the capsule endoscope 2 when the capsule endoscope 2 is introduced into the subject H twice are acquired.

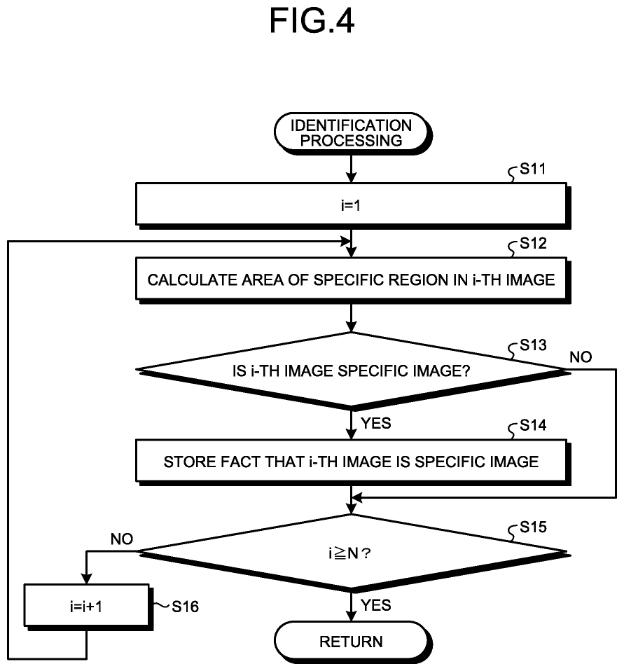

Next, the identification unit 54 performs identification processing on the first image group (Step S2). FIG. 4 is a flowchart illustrating the identification processing illustrated in FIG. 3. As illustrated in FIG. 4, the control unit 57 sets a variable i so that i=1 (Step S11).

Then, the first calculation unit 541 calculates the amount (the area, the number of pixels, or the like) of a specific region included in the i-th image (Step S12).

Next, the first identification unit 542 determines whether or not the i-th image is a specific image in which the amount of the specific region is equal to or more than a predetermined threshold value (equal to or more than a predetermined area) stored in the storage unit 52. (Step S13). The specific image is an image including a region that does not include a captured image of the subject H (inner wall of the gastrointestinal tract) in an amount that is equal to or more than a predetermined threshold value due to the specific region such as a bubble, residue, or noise. The threshold value may be a value input by the user.

In a case where the i-th image is the specific image (Step S13: Yes), the control unit 57 stores, in the storage unit 52, the fact that the i-th image is the specific image (Step S14).

On the other hand, in a case where the i-th image is not the specific image (Step S13: No), the processing directly proceeds to Step S15.

Next, the control unit 57 determines whether or not the variable i is equal to or more than the number N of all images (Step S15).

In a case where the variable i is smaller than N (Step S15: No), the control unit 57 increments the variable i (i=i+1) (Step S16), and returns to Step S12 to continue the processing. On the other hand, in a case where the variable i is N or more (Step S15: Yes), the identification processing ends.

By the identification processing described above, a region of the subject H that is not captured by the capsule endoscope 2 in the first image group is identified. Specifically, a region between the specific images that are consecutive in time series is the region of the subject H that is not captured by the capsule endoscope 2.

Returning to FIG. 3, the identification unit 54 performs identification processing on the second image group (Step S3). As a result, a region of the subject H that is not captured by the capsule endoscope 2 in the second image group is identified.

Then, the first specifying unit 55 specifies an overlapping section of the subject H in which the region identified by the identification unit 54 in each of the first and second image groups overlap each other between the first and second image groups (Step S4).

Next, the generation unit 56 calculates a distance from a reference position to the overlapping section (Step S5).

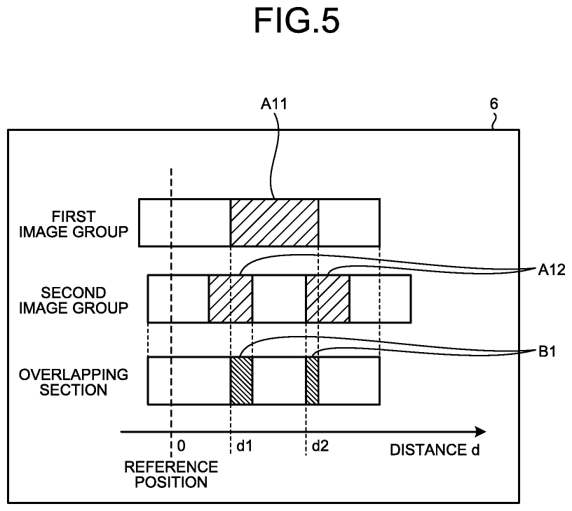

Further, the display controller causes the display device 6 to display an image displaying the distance to the overlapping section (Step S6). FIG. 5 is a diagram illustrating an example of the image displayed on the display device. As illustrated in FIG. 5, the display device 6 displays the first image group, the second image group, and the overlapping section. A horizontal axis of FIG. 5 represents a distance in a forward direction from the mouth of the subject H toward the anus. Further, the first image group and the second image group are arranged so that reference positions indicated by a broken line match. The reference position is, for example, a site such as the mouth, the cardia, the pylorus, the ileum, or the anus, or a lesion such as a hemostasis site or a ridge site. The reference position may be detected from an image, or the user may observe the image to select the reference position.

In the first image group, the region of the subject H that is not captured by the capsule endoscope 2 is a region A11. Similarly, in the second image group, the region of the subject H that is not captured by the capsule endoscope 2 is a region A12. The region A11 and the region A12 are identified by the identification unit 54. Then, the first specifying unit 55 specifies an overlapping section B1 as a section in which the region A11 and the region A12 overlap each other. Further, the display device 6 displays a distance d1 and a distance d2 as the distances from the reference positions generated by the generation unit 56 to the overlapping section.

The user can recognize a section of the subject H that is not captured by the capsule endoscope 2 even after performing an examination multiple times, due to the overlapping section B1 displayed on the display device 6. As a result, the user can easily specify a lesion such as a bleeding source by selectively examining the overlapping section B1 with a small intestine endoscope or the like.

In the examination using the capsule endoscope 2, in a case of a patient with obscure gastrointestinal bleeding (OGIB), in which a bleeding source is not found by the examination using the capsule endoscope 2 and anemia is not alleviated, the bleeding source is specified by repeatedly performing the examination using the capsule endoscope 2. However, in a case where the bleeding source is in a region where the capsule endoscope 2 passes through quickly or in a region where residues are likely to accumulate, the bleeding source may not be found even after performing the examination using the capsule endoscope 2 multiple times. In such a case, the image processing apparatus 5 automatically specifies the overlapping section B1 which is the section of the subject H that is not captured by the capsule endoscope 2 in the examination performed multiple times. As a result, the user can easily specify the bleeding source by examining the overlapping section B1 with a small intestine endoscope or the like.

### MODIFIED EXAMPLE 1-1

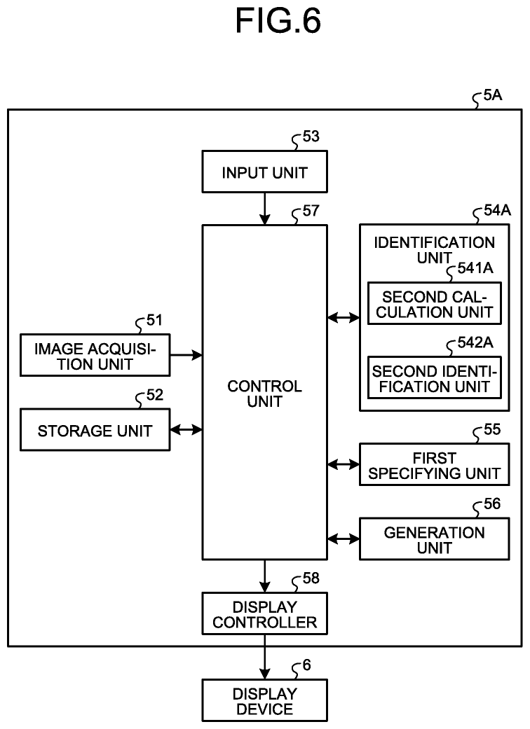

FIG. 6 is a block diagram illustrating an image processing apparatus according to Modified Example 1-1. As illustrated in FIG. 6, an identification unit 54A of an image processing apparatus 5A includes a second calculation unit 541A that calculates the amount of change in a parameter based on a position of the capsule endoscope 2 when at least two images of an image group are captured, and a second identification unit 542A that identifies a region of the subject H that is not captured by the capsule endoscope 2 on the basis of the amount of change calculated by the second calculation unit 541A. The amount of change is an amount that is determined based on the degree of similarity between the at least two images, or on the position, speed, or acceleration of the capsule endoscope. Note that the position of the capsule endoscope 2 can be detected from information acquired by the receiving device 3. Further, the speed or acceleration of the capsule endoscope 2 can be acquired from a speed sensor or an acceleration sensor embedded in the capsule endoscope 2.

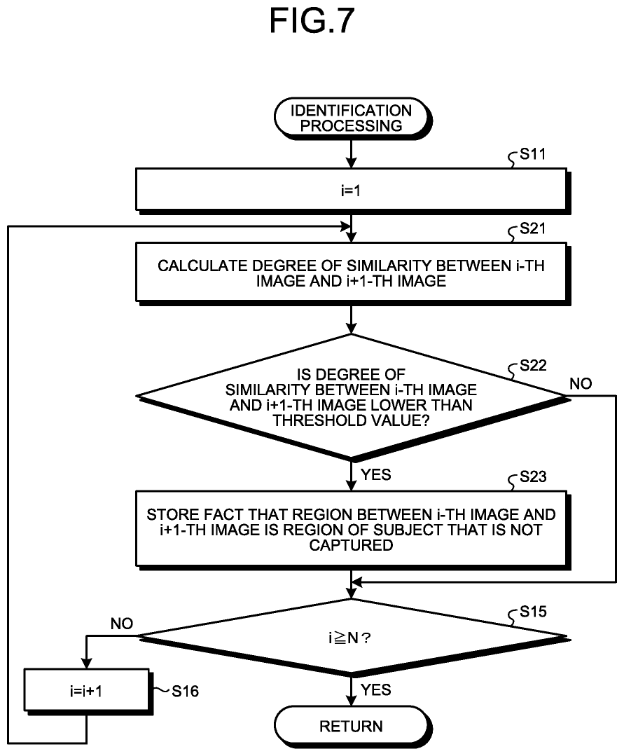

Next, an operation of the image processing apparatus 5A will be described. The operation of the image processing apparatus 5A differs from the image processing apparatus 5 only in identification processing. FIG. 7 is a flowchart illustrating identification processing of the image processing apparatus illustrated in FIG. 6. As illustrated in FIG. 7, after performing the processing in Step S11 in the same manner as in the first embodiment, the second calculation unit 541A calculates the degree of similarity between the i-th image and the i+1-th image of an image group that are arranged in time series (Step S21).

Then, the second identification unit 542A identifies whether or not the degree of similarity calculated by the second calculation unit 541A is lower than a predetermined threshold value (Step S22). Note that the threshold value may be a value stored in a storage unit 52 in advance, or may be a value input by the user. In a case where it is identified by the second identification unit 542A that the degree of similarity is lower than the predetermined threshold value (Step S22: Yes), a control unit 57 stores, in a storage unit 52, the fact that a region between the i-th image and the i+1-th image is a region of the subject H that is not captured by the capsule endoscope 2 (Step S23).

On the other hand, in a case where it is identified by the second identification unit 542A that the degree of similarity is equal to or higher than the predetermined threshold value (Step S22: No), the processing directly proceeds to Step S15.

Next, the processing in Steps S15 and S16 is performed in the same manner as in the first embodiment.

As in Modified Example 1-1, the identification unit 54 may identify a region of the subject H that is not captured by the capsule endoscope 2 by using an amount that is determined based on the degree of similarity between at least two images, or on a position, speed, or acceleration of the capsule endoscope.

### MODIFIED EXAMPLE 1-2

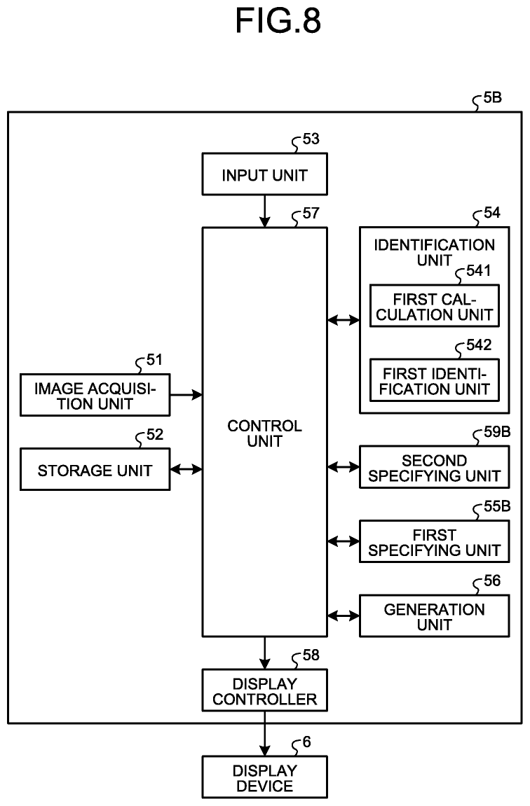

FIG. 8 is a block diagram illustrating an image processing apparatus according to Modified Example 1-2. As illustrated in FIG. 8, an image processing apparatus 5B includes a second specifying unit 59B that specifies, as a reciprocating image group, a group of reciprocating images captured when the capsule endoscope 2 reciprocates in the subject H, in each of a plurality of image groups. The second specifying unit 59B specifies the reciprocating image group by comparing consecutive images arranged in time series and detecting a direction in which the capsule endoscope 2 moves. Alternatively, the second specifying unit 59B may specify the reciprocating image group on the basis of position information of the capsule endoscope 2 received by the receiving device 3, a capturing time, an image number, or a speed or acceleration measured by a speed sensor or acceleration sensor embedded in the capsule endoscope 2.

A first specifying unit 55B of the image processing apparatus 5B specifies, in the reciprocating image group, a section of the subject H that is overlappingly identified, by an identification unit 54, as a region of the subject H that is not captured by the capsule endoscope 2 when the capsule endoscope 2 reciprocates in the subject H.

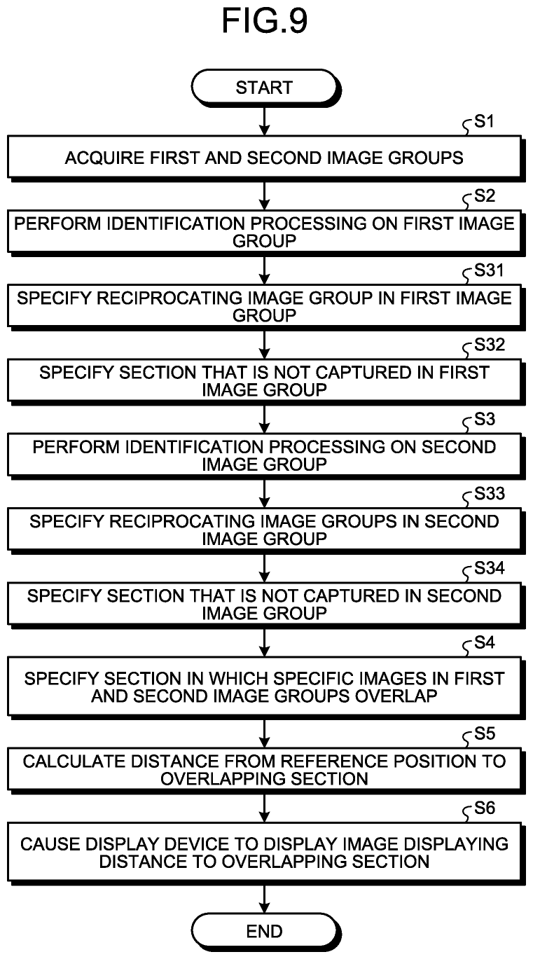

Next, an operation of the image processing apparatus 5B will be described. FIG. 9 is a flowchart illustrating an operation of the image processing apparatus illustrated in FIG. 8. As illustrated in FIG. 9, after performing the processing in Steps S1 and S2 in the same manner as in the first embodiment, the second specifying unit 59B specifies the reciprocating image group in the first image group (Step S31).

FIG. 10 is a diagram illustrating the reciprocating image group. In FIG. 10, a direction toward the right side of the paper is a forward direction. The forward direction is a direction in which the capsule endoscope 2 advances from the mouth of the subject H toward the anus. The second specifying unit 59B compares consecutive images arranged in time series in the first image group, and identifies a direction in which the capsule endoscope 2 moves when each image is captured. In FIG. 10, the capsule endoscope 2 advances in the forward direction in sections s1, s21, s23, and s3, and the capsule endoscope 2 advances in a backward direction in a section s22. At this time, the second specifying unit 59B specifies the sections s21, s22, and s23 as the reciprocating image group.

Next, the first specifying unit 55B specifies a section of the subject H that is not captured by the capsule endoscope 2 in the first image group (Step S32).

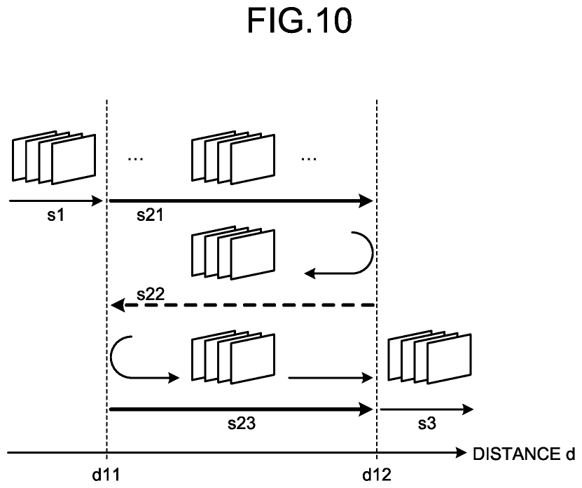

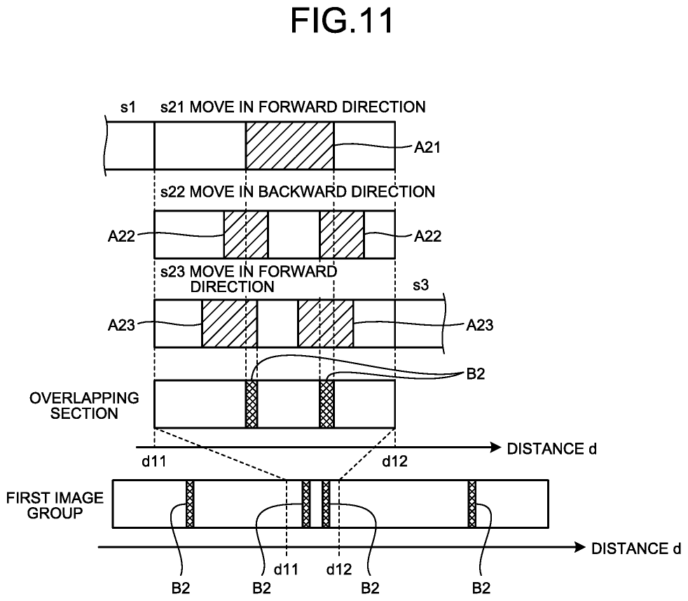

FIG. 11 is a diagram illustrating a state in which an overlapping section is specified from the reciprocating image group. As illustrated in FIG. 11, the first specifying unit 55B specifies a section of the subject H that is overlappingly identified, by an identification unit 54, as a region of the subject H that is not captured by the capsule endoscope 2 when the capsule endoscope 2 reciprocates in the subject H, in the first image group. Specifically, the first specifying unit 55B specifies, as an overlapping section B2, a section in which a region A21 of the subject H that is not captured by the capsule endoscope 2 in the section s21, a region A22 of the subject H that is not captured by the capsule endoscope 2 in the section s22, and a region A23 of the subject H that is not captured by the capsule endoscope 2 in the section s23 overlap each other, the regions A21, A22, and A23 being identified by the identification unit 54.

Further, as illustrated in FIG. 11, the first specifying unit 55 specifies the overlapping section for images other than the reciprocating image group as in the first embodiment, and specifies the overlapping section B2 for the entire first image group.

Then, as in Steps S2, S31, and S32, in Steps S3, S33, and S34, an overlapping section of the second image group is specified. Then, the processing in Steps S4 to S6 is performed in the same manner as in the first embodiment, and a series of treatments ends.

According to Modified Example 1-2, a section that is not captured by the capsule endoscope 2 even once when the capsule endoscope 2 reciprocates is specified as the overlapping section B2. Therefore, sections that the user examines again by using a small intestine endoscopy are reduced, and the burden on the user can be reduced.

### Second Embodiment

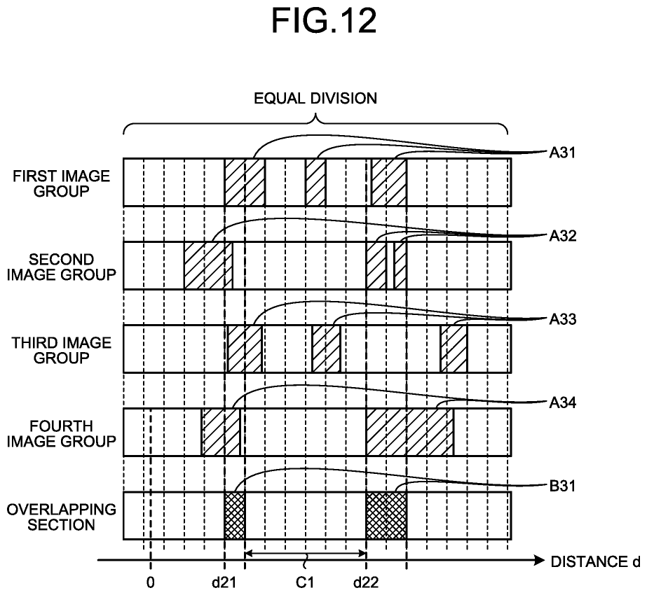

A configuration of an image processing apparatus 5 according to a second embodiment is the same as that of the first embodiment, and the second embodiment differs from the first embodiment only in processing in the image processing apparatus 5. FIG. 12 is a diagram illustrating a state in which the image processing apparatus according to the second embodiment specifies overlapping sections. As illustrated in FIG. 12, a first specifying unit 55 of the image processing apparatus 5 performs normalization of each of acquired first to fourth image groups into position series in an entire section, and divides the entire section of each of the plurality of image groups into sections with an equal distance D.

An identification unit 54 identifies regions A31 to A34 of the subject H that are not captured by the capsule endoscope 2, in each of the plurality of image groups.

The first specifying unit 55 identifies whether or not each section of each of the plurality of image groups includes the region identified by the identification unit 54. Then, the first specifying unit 55 specifies overlapping sections B31 in which an overlapping proportion of the regions identified by the identification unit 54 is 75% or more.

A generation unit 56 calculates a distance d21 and a distance d22 as information regarding positions of the overlapping sections B31. An image including a captured image of the pylorus in the fourth image group is a position where distance d=0, the position corresponding to a reference position, and the distance d21 and the distance d22 are distances from the reference position to the overlapping sections B31. Further, the generation unit 56 calculates a distance C1 between the two overlapping sections B31 as information regarding the positions of the overlapping sections B31.

FIG. 12 illustrates a case where the overlapping sections B31 include one section in which a proportion of a region identified by the identification unit 54 on the basis of the distance d21 is 100%, and two sections in which a proportion of a region identified by the identification unit 54 on the basis of the distance d22 is 75%. Since the distance d21 and the distance d22 are displayed on the display device 6, the user can know a distance to a region to be examined by using a small intestine endoscope or the like. Further, since the distance C1 is displayed on the display device 6, the user can easily perform movement to another overlapping section B31 after examining the first overlapping section B31.

### MODIFIED EXAMPLE 2-1

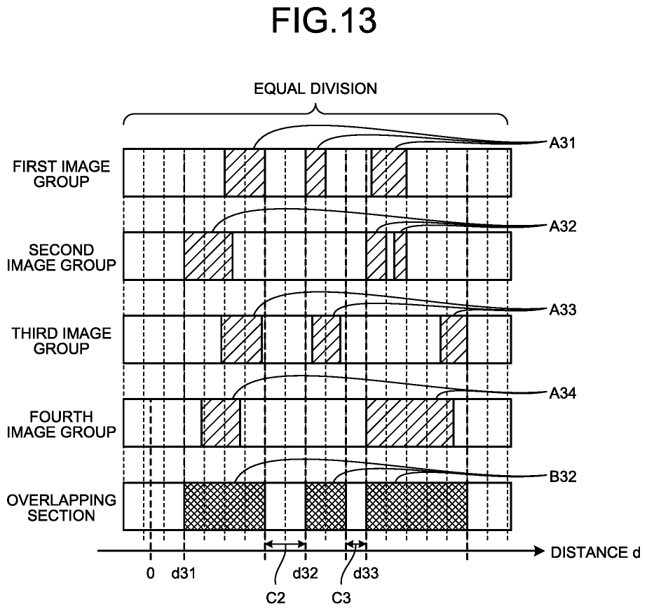

FIG. 13 is a diagram illustrating a state in which an image processing apparatus according to Modified Example 2-1 specifies overlapping sections. As illustrated in FIG. 13, a first specifying unit 55 may specify, as an overlapping section B32, a section including at least one region identified by an identification unit 54.

A generation unit 56 calculates a distance d31, a distance d32, and a distance d33 as information regarding positions of the overlapping sections B32. An image including a captured image of the pylorus in a fourth image group is a position where distance d=0, the position corresponding to a reference position, and the distance d31, the distance d32, and the distance d33 are distances from the reference position to the overlapping sections B32. Further, the generation unit 56 calculates, as the information regarding the positions of the overlapping sections B32, a distance C2 between the first overlapping section B32 and the second overlapping section B32, and a distance C3 between the second overlapping section B32 and the third overlapping section B32.

FIG. 13 illustrates a case where the overlapping sections B32 include four sections each including a region identified by the identification unit 54 on the basis of the distance d31, two sections each including a region identified by the identification unit 54 on the basis of the distance d32, and five sections each including a region identified by the identification unit 54 on the basis of the distance d33.

### Third Embodiment

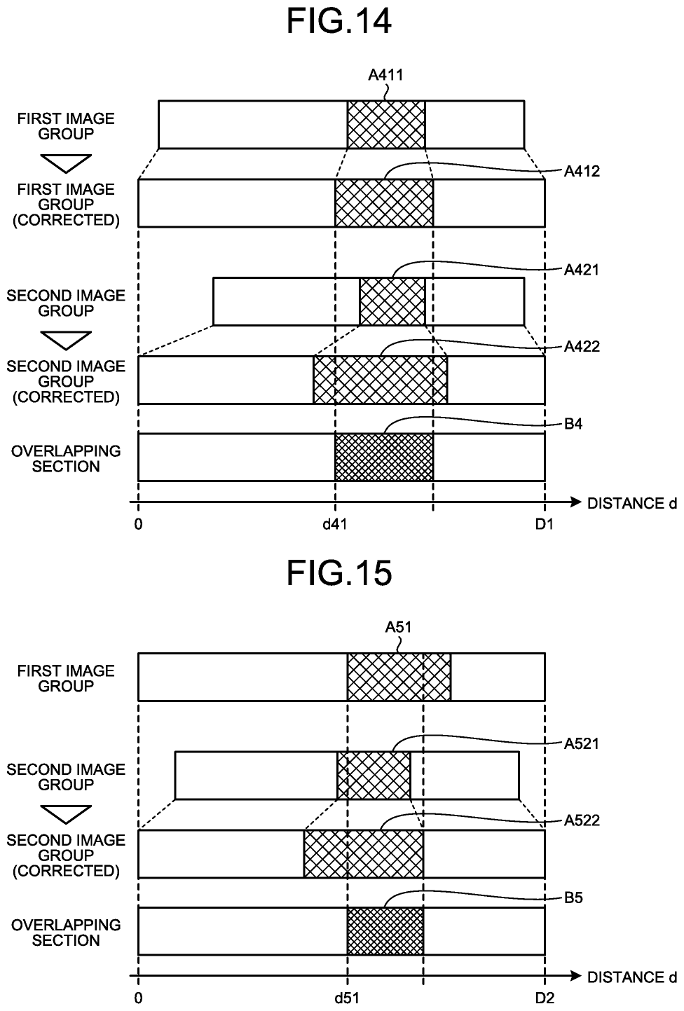

FIG. 14 is a diagram illustrating a state in which an image processing apparatus according to a third embodiment specifies an overlapping section. As illustrated in FIG. 14, a generation unit 56 corrects a position of each image in a first image group so that the first captured image in the first image group and the last captured image in the first image group correspond to a predetermined distance d=0 and a distance d=D1, respectively. By this correction, a region A411 identified by an identification unit 54 as a region of the subject H that is not captured by the capsule endoscope 2 in the first image group is corrected to a region A412.

Similarly, the generation unit 56 corrects a position of each image in a second image group so that the first captured image in the second image group and the last captured image in the second image group correspond to the predetermined distance d=0 and the distance d=D1, respectively. By this correction, a region A421 identified by the identification unit 54 as a region of the subject H that is not captured by the capsule endoscope 2 in the second image group is corrected to a region A422.

Then, a first specifying unit 55 specifies, as an overlapping section B4, a section in which the region A412 and the region A422 overlap each other.

### MODIFIED EXAMPLE 3-1

FIG. 15 is a diagram illustrating a state in which an image processing apparatus according to Modified Example 3-1 specifies overlapping sections. As illustrated in FIG. 15, the first captured image and the last captured image in a first image group correspond to a distance d=0 and a distance d=D2, respectively. Then, a generation unit 56 corrects a position of each image in a second image group so that the first captured image in the second image group and the last captured image in the second image group correspond to the predetermined distance d=0 and the distance d=D2, respectively. By this correction, a region A521 identified by an identification unit 54 as a region of the subject H that is not captured by the capsule endoscope 2 in the second image group is corrected to a region A522.

Then, a first specifying unit 55 specifies, as an overlapping section B5, a section in which a region A51 and the region A522 overlap each other.

### MODIFIED EXAMPLE 3-2

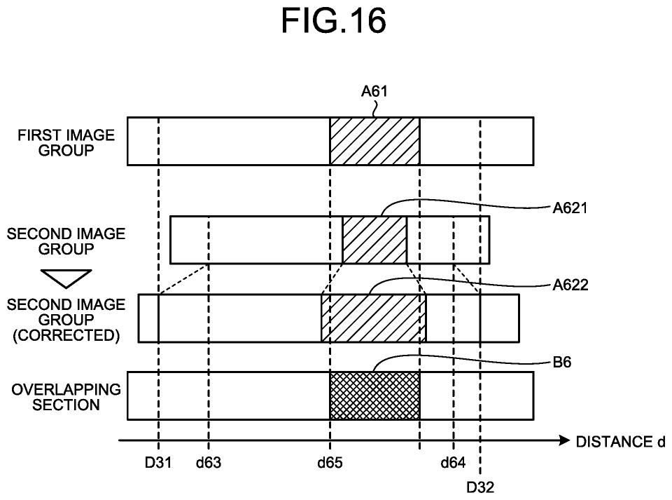

FIG. 16 is a diagram illustrating a state in which an image processing apparatus according to Modified Example 3-2 specifies overlapping sections. As illustrated in FIG. 16, an image corresponding to the pylorus and an image corresponding to the ileocecal valve in a first image group correspond to a distance d=D31 and a distance d=D32, respectively. Then, a generation unit 56 corrects a position of each image in a second image group so that the image corresponding to the pylorus in the second image group and the image corresponding to the ileocecal valve in the second image group correspond to the predetermined distance d=D31 and the distance d=D32, respectively. By this correction, a region A621 identified by an identification unit 54 as a region of the subject H that is not captured by the capsule endoscope 2 in the second image group is corrected to a region A622.

Then, a first specifying unit 55 specifies, as an overlapping section B6, a section in which a region A61 and the region A622 overlap each other.

Note that three or more reference positions may be set to sites such as the mouth, the cardia, the pylorus, the ileum, and the anus, or lesions such as a hemostasis site and a ridge site, and different corrections may be applied for the respective reference positions. Further, the reference position may be detected from an image, or the user may observe the image to select the reference position.

### Fourth Embodiment

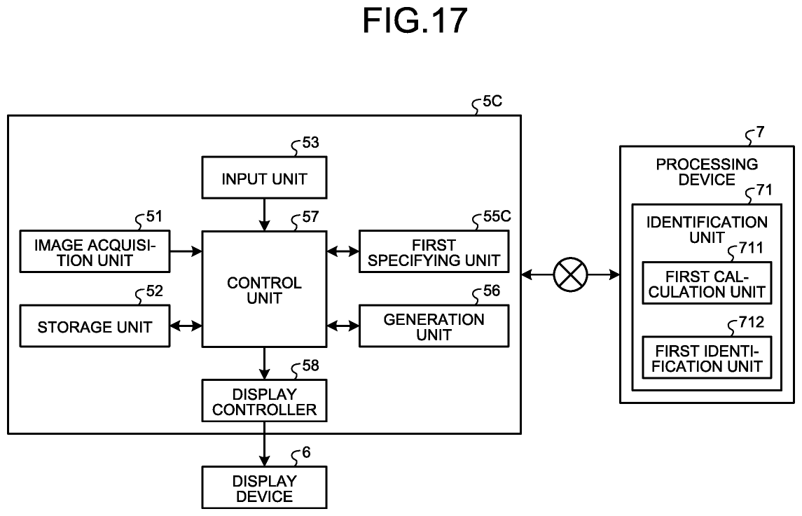

FIG. 17 is a block diagram illustrating an image processing apparatus according to a fourth embodiment. As illustrated in FIG. 17, a processing device 7 is connected to an image processing apparatus 5C. The processing device 7 is a server connected via an internet line, or the like. The processing device 7 includes an identification unit 71. The identification unit 71 includes a first calculation unit 711 and a first identification unit 712. Functions of the identification unit 71, the first calculation unit 711, and the first identification unit 712 are the same as those of the identification unit 54, the first calculation unit 541, and the first identification unit 542 of the image processing apparatus 5. Therefore, a description thereof will be omitted. Meanwhile, the image processing apparatus 5C does not include the identification unit, the first calculation unit, and the first identification unit.

A first specifying unit 55C acquires a region of the subject H that is not captured by the capsule endoscope 2, the regions being identified in each of a plurality of image groups on the basis of a characteristic of each of the plurality of image groups, and specifies a section of the subject H in which the region in each of a plurality of image groups overlap each other between the plurality of image groups. In other words, the first specifying unit 55C specifies a section of the subject H in which the region identified by the identification unit 71 in each of the plurality of image groups overlaps each other between the plurality of image groups. However, the first specifying unit 55C may specify at least one section of the subject H in which the region is included in one of the plurality of image groups.

As in the fourth embodiment described above, the image processing apparatus 5C does not include the identification unit, the first calculation unit, and the first identification unit, and the processing device 7 connected via the Internet may perform processing that is to be performed by the identification unit. Similarly, the processing that is to be performed by the identification unit may be performed on a cloud including a plurality of processing devices (server group).

### MODIFIED EXAMPLE 4-1

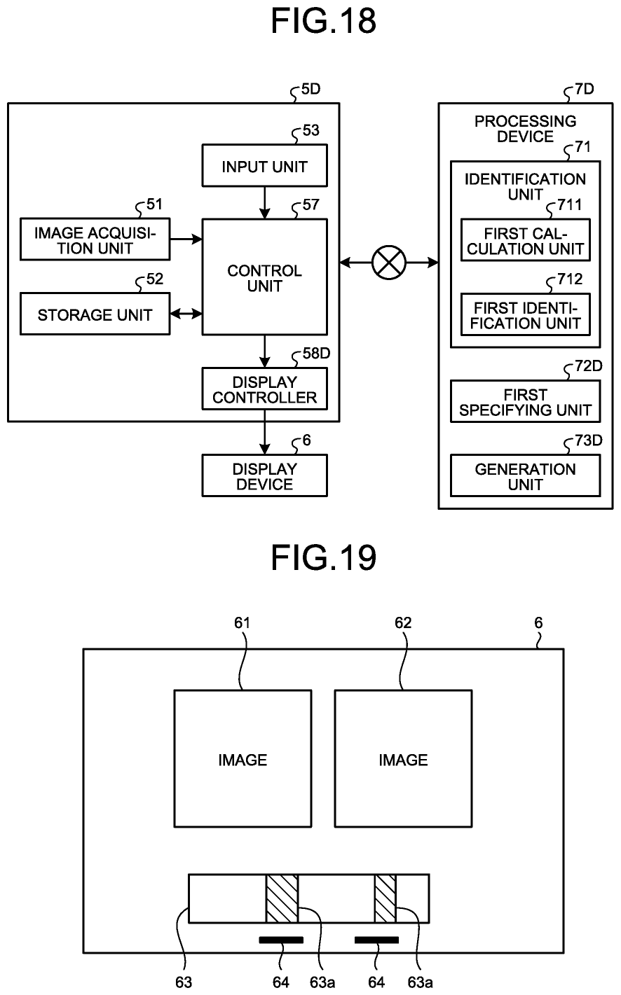

FIG. 18 is a block diagram illustrating an image processing apparatus according to Modified Example 4-1. As illustrated in FIG. 18, a processing device 7D is connected to an image processing apparatus 5D. The processing device 7D includes an identification unit 71, a first specifying unit 72D, and a generation unit 73D. Functions of the identification unit 71, the first specifying unit 72D, and the generation unit 73D are the same as those of the identification unit 54, the first specifying unit 55, and the generation unit 56 of the image processing apparatus 5, and thus a description thereof will be omitted. Meanwhile, the image processing apparatus 5D does not include the identification unit, the first specifying unit, and the generation unit.

A display controller 58D acquires a specified section of the subject H in which a region of the subject H that is not captured by the capsule endoscope 2 in each of a plurality of image groups overlaps each other between a plurality of image groups, the region being identified in each of a plurality of image groups on the basis of a characteristic of each of the plurality of image groups, and causes the display device 6 to display information regarding a position of the section. In other words, the first specifying unit 72D specifies the section of the subject H in which the regions identified by the identification unit 71 overlap each other between the plurality of image groups, the generation unit 73D generate the information regarding the position of the section specified by the first specifying unit 72D, and the display controller 58D causes the display device 6 to display the information regarding the position of the section. However, the first specifying unit 72D may specify at least one section of the subject H in which the region is included in one of the plurality of image groups.

As in Modified Example 4-1 described above, the image processing apparatus 5D does not include the identification unit, the first specifying unit, and the generation unit, and the processing device 7D connected via the Internet may perform processing that is to be performed by the identification unit, the first specifying unit, and the generation unit, respectively. Similarly, the processing that is to be performed by the identification unit, the first specifying unit, and the generation unit may be performed on a cloud including a plurality of processing devices (server group).

### Fifth Embodiment

FIG. 19 is a diagram illustrating an example of the image displayed on the display device. As illustrated in FIG. 19, in the display device 6, images 61 and 62, a distance bar 63 indicating a region 63a that is not captured by the capsule endoscope 2 in a current examination, and a marker 64 indicating a region that is not captured by the capsule endoscope 2 in a past examination.

As such, only a current examination result may be displayed by the distance bar 63, and a past examination result may be displayed by the marker 64. Note that in a case where there are a plurality of past examination results, markers for each examination may be displayed side by side. In addition, in a case where there are a plurality of past examination results, a marker indicating a region that is repeatedly not captured by the capsule endoscope 2 in the past examinations may be displayed. Similarly, in a case where there are a plurality of past examination results, a marker indicating a region including a portion that is repeatedly not captured the capsule endoscope 2 in the past examinations may be displayed, the portion having a predetermined proportion or more. In addition, in a case where there are a plurality of past examination results, a marker indicating a region that is not captured by the capsule endoscope 2 even once in the past examinations may be displayed.

### MODIFIED EXAMPLE 5-1

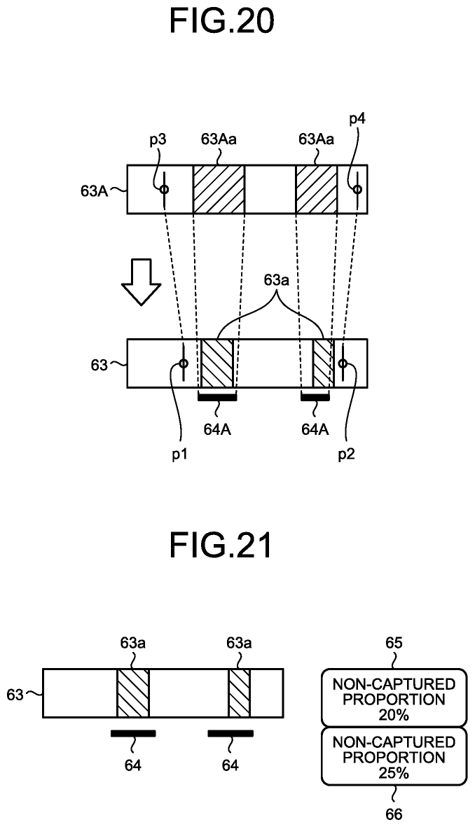

FIG. 20 is a diagram illustrating a state in which reference positions match each other. As illustrated in FIG. 20, a distance bar 63A for a past examination may be corrected on the basis of a distance bar 63 for a current examination, and displayed on the display device 6. Specifically, the distance bar 63A for the past examination may be corrected so that a reference position p3 and a reference position p4 in the past examination corresponding to a reference position p1 and a reference position p2 of the current examination, respectively, overlap with the reference position pl and the reference position p2 of the current examination, respectively. Here, a region 63Aa that is not captured by the capsule endoscope 2 in the past examination is corrected to a marker 64A.

### MODIFIED EXAMPLE 5-2

FIG. 21 is a diagram illustrating a state in which a non-captured proportion is displayed. As illustrated in FIG. 21, in a current examination and a past examination, proportions (non-captured proportions) of regions that are not captured by the capsule endoscope 2 may be displayed by using icons 65 and 66 each including a numerical value. Note that in a case where there are a plurality of past examination results, icons of non-captured proportions for each examination may be displayed side by side. In addition, in a case where there are a plurality of past examination results, a proportion of a region that is repeatedly not captured by the capsule endoscope 2 in the past examinations may be displayed in a form of a numerical value. Similarly, in a case where there are a plurality of past examination results, a proportion of a region including a portion that is repeatedly not captured the capsule endoscope 2 in the past examinations may be displayed in a form of a numerical value, the portion having a predetermined proportion or more. In addition, in a case where there are a plurality of past examination results, a proportion of a region that is not captured by the capsule endoscope 2 even once in the past examinations may be displayed in a form of a numerical value.

### MODIFIED EXAMPLE 5-3

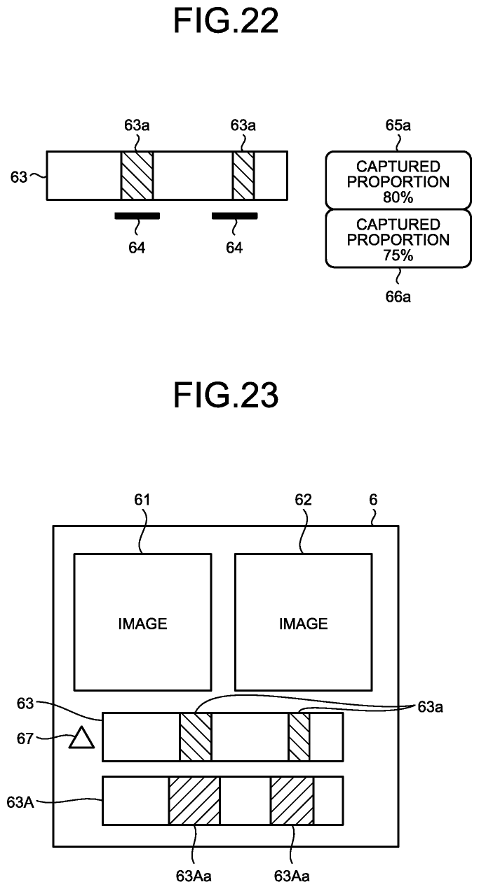

FIG. 22 is a diagram illustrating a state in which a captured proportion is displayed. As illustrated in FIG. 22, in a current examination and a past examination, proportions (captured proportions) of regions captured by the capsule endoscope 2 may be displayed by using icons 65a and 66a each including a numerical value.

### MODIFIED EXAMPLE 5-4

FIG. 23 is a diagram illustrating a state in which distance bars are displayed side by side. As illustrated in FIG. 23, a distance bar 63 for a current examination and a distance bar 63A for a past examination may be displayed side by side. Further, the distance bar 63A for the past examination may be hidden by clicking a button 67.

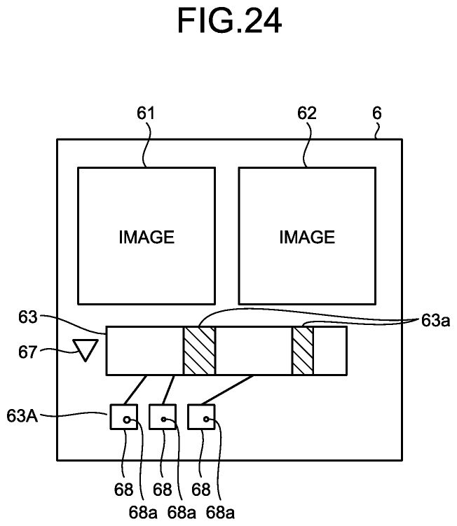

FIG. 24 is a diagram illustrating a state in which a distance bar is hidden. As illustrated in FIG. 24, when the button 67 is clicked, the distance bar 63A for the past examination is hidden. Here, captured images 68 may be displayed in a region where the distance bar 63A for the past examination was displayed. The captured images 68 are each an image including a reddish (bleeding) 68a and the like, the image being particularly noticed by the user, and is an image selected by the user from an image group and saved. Each captured image 68 is an image displayed at a position connected to the distance bar 63 by a straight line.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing apparatus that performs image processing on an image captured by a capsule endoscope introduced into a subject, the image processing apparatus comprising:
an identification circuit configured to
calculate a characteristic of each of a plurality of image groups captured when the capsule endoscope is introduced into the subject multiple times, each image group being a group of images that are captured each time the capsule endoscope is introduced into the subject, and
identify, based on the calculated characteristic, a first region or a second region in each image group, the first region being a region that does not include an image of the subject captured by the capsule endoscope, the second region being a region that is regarded as not including the captured image of the subject; and
a first specifying circuit configured to specify at least one section of the subject in the plurality of image groups, the at least one section including the first region or the second region.

2. The image processing apparatus according to claim 1, wherein the first specifying circuit is configured to specify the section of the subject in which an overlapping proportion of the first region or the second region in the image group overlapping each other between the plurality of image groups is equal to or more than a predetermined value.

3. The image processing apparatus according to claim 1, wherein
the identification circuit includes:
a first calculation circuit configured to calculate an amount of a specific region in each image included in each of the plurality of image groups; and
a first identification circuit configured to identify the second region based on the amount of the specific region.

4. The image processing apparatus according to claim 3, wherein
the first identification circuit is configured to
identify whether or not each image is a specific image in which the amount of the specific region is equal to or more than a predetermined threshold value, and
identify, as the second region, a region between specific images that are consecutive in time series.

5. The image processing apparatus according to claim 1, wherein
the identification circuit includes:
a second calculation circuit configured to calculate an amount of change in a parameter based on a position of the capsule endoscope when at least two images of each of the plurality of image groups are captured; and
a second identification circuit configured to identify the first region based on the amount of change.

6. The image processing apparatus according to claim 3, wherein the specific region is a region including a captured image of a bubble, a residue, or noise.

7. The image processing apparatus according to claim 5, wherein the amount of change is an amount determined based on a degree of similarity between the at least two images, or on a position, speed, or acceleration of the capsule endoscope.

8. The image processing apparatus according to claim 1, further comprising:
a generation circuit configured to generate information regarding a position of the at least one section; and
a display controller configured to cause a display to display the information.

9. The image processing apparatus according to claim 8, wherein the information is a distance from a reference position in the subject to the section.

10. The image processing apparatus according to claim 8, wherein the at least one section is a plurality of sections, and the information is a distance between the plurality of sections.

11. The image processing apparatus according to claim 1, further comprising
a second specifying circuit configured to specify a group of reciprocating images captured when the capsule endoscope reciprocates in the subject, in each of the plurality of image groups, wherein
the first specifying circuit configured to specify, in the group of the reciprocating images, a section of the subject that is overlappingly identified as the first region or the second region when the capsule endoscope reciprocates in the subject.

12. The image processing apparatus according to claim 1, wherein
the at least one section is a plurality of sections, and
the image processing apparatus further comprises a display controller configured to cause a display to display the plurality of sections side by side.

13. A capsule endoscope system comprising:
the image processing apparatus according to claim 1; and
the capsule endoscope.

14. A method of operating an image processing apparatus that performs image processing on an image captured by a capsule endoscope introduced into a subject, the method comprising:
calculating, by an identification circuit, a characteristic of each of a plurality of image groups captured when the capsule endoscope is introduced into the subject multiple times, each image group being a group of images that are captured each time the capsule endoscope is introduced into the subject;
identifying, based on the calculated characteristic, a first region or a second region in each image group, the first region being a region that does not include an image of the subject captured by the capsule endoscope, the second region being a region that is regarded as not including the captured image of the subject; and
specifying, by a first specifying circuit, at least one section of the subject in the plurality of image groups, the at least one section including the first region or the second region.

15. A non-transitory computer-readable recording medium on which an executable program is recorded, the program instructing an image processing apparatus that performs image processing on an image captured by a capsule endoscope introduced into a subject to execute:
calculating, by an identification circuit, a characteristic of each of a plurality of image groups captured when the capsule endoscope is introduced into the subject multiple times, each image group being a group of images that are captured each time the capsule endoscope is introduced into the subject,
identifying, based on the calculated characteristic, a first region or a second region in each image group, the first region being a region that does not include an image of the subject captured by the capsule endoscope, the second region being a region that is regarded as not including the captured image of the subject; and
specifying, by a first specifying circuit, at least one section of the subject in the plurality of image groups, the at least one section including the first region or the second region.
